(19) United States
(12) Patent Application Publication
(10) Pub. No.: US 2024/0131252 A1
(43) Pub. Date: Apr. 25, 2024

(54) PUMPS FOR IMPROVING FLUID DELIVERY AND METHODS OF USE THEREOF

(71) Applicant: CareFusion 303, Inc., San Diego, CA (US)

(72) Inventor: Archana NAGARAJA RAO, Los Angeles, CA (US)

(21) Appl. No.: 18/489,691

(22) Filed: Oct. 17, 2023

Related U.S. Application Data
(60) Provisional application No. 63/418,901, filed on Oct. 24, 2022.

(51) Int. Cl. A61M 5/142; A61M 5/14

(52) U.S. Cl. CPC A61M 5/1424; A61M 5/1411

(57) ABSTRACT

IV sets including a plurality of hand pumps and methods of use thereof are described herein. An IV set includes a first portion of tubing, a second portion of tubing, and a drip chamber. The drip chamber includes a spikeless inlet fluidically coupled to the first portion of the tubing, an outlet fluidic ally coupled to the second portion of the tubing, and a curved drop former positioned within the drip chamber between the inlet and the outlet. The curved drop former is configured to receive a fluid via the spikeless inlet and form, based on a curvature of its surface, one or more droplets having a substantially uniform consistency and flowing at a substantially constant rate through the drip chamber.

### CROSS REFERENCE TO RELATED APPLICATION

This application claims the benefit of priority under 35 U.S.C. § 119 to U.S. Provisional Patent Application Ser. No. 63/418,901, entitled “A PLURALITY OF HAND PUMPS FOR USE WITH AN IV SET AND METHODS OF USE THEREOF”, filed on Oct. 24, 2022, the entire contents of which is incorporated herein by reference.

### FIELD OF THE INVENTION

The present disclosure generally relates to medical fluid distribution systems, and, in particular, to IV sets.

### BACKGROUND

Medical treatments often include the infusion of a medical fluid (e.g., a saline solution or a liquid medication) to patients using an IV set that is used to distribute the medical fluid to a patient. Existing IV sets can provide inconsistent flow rates and generate blockages that reduce flow rates and reduce the efficacy of patient treatment. Clinicians are often required to interact with an IV set to remove blockages within the IV set. Due to the design of existing IV sets, clinicians can become fatigued due to the constant need to reach and compress a hand pump or other components of the IV set.

As such, there is a need for an IV set that provides faster flow rates and reduces the number of blockages and, when blockages do occur, provides clinicians with a convenient and easy way to remove blockages and/or adjust a flow rate of medical fluid provided to a patient.

### SUMMARY

The disclosed subject matter relates to different components of an IV set. In certain implementations, the different components of the IV set include one or more improvements configured to improve design factors that contribute to the usability of a hand pump of the IV set as well as the material stiffness of the hand pump. In certain implementations, the different components of the IV set are configured to increase the volume of fluid delivered to a patient as well as the rate at which fluid is delivered to a patient, which improves patient outcomes, reduces clinician fatigue, and reduces the amount of clinician interference needed. In certain implementations, the different components of the IV set are configured to improve fluid volume and flow rates, increase filtration speeds further increasing flow rates, reduce the coefficient of friction reducing the residence time, as well as improve compatibility of the IV set with patient by using blood compatible materials. The different components of the IV set are configured to improve the efficiency of fluid delivery by reducing the number of blockages or flow interference. A non-exhaustive list of improvements to the different components of the IV set include the use of materials (e.g., thermoplastic elastomers) and coatings (e.g., hydrophobic coatings) that contribute to the high flow rates across the different components of the IV set; changes to tubing of the IV set (e.g., inner diameter of the tubing); increased filter size and surface area; increased size of a drip chamber of the IV set, and a number of other improvements described below.

In accordance with some implementations, an IV set is provided. The IV set includes a first portion of tubing, a second portion of tubing and a drip chamber. The drip chamber includes a spikeless inlet fluidically coupled to the first portion of the tubing, an outlet fluidically coupled to the second portion of the tubing, and a curved drop former positioned within the drip chamber between the inlet and the outlet. The curved drop former is configured to receive a fluid via the spikeless inlet and form, based on a curvature of its surface, one or more droplets having a substantially uniform consistency and flowing at a substantially constant rate through the drip chamber (e.g., such that a flow rate of the IV set can be visually monitored by a visual indicator provided through the drip chamber). In some implementations, the curved drop former includes a hydrophobic coating. In some implementations, a body of the drip chamber includes a hydrophobic coating that forms a predetermined contact angle such that the one or more droplets are uniform and consistent.

In some implementations, the drip chamber is formed an antistatic material. In some implementations, the drip chamber comprises a resilient material such that the drip chamber rebounds to an uncompressed state after it is compressed. In some implementations, the drip chamber further includes a filter of a predetermined surface area fluidically coupled to the outlet such that the filter captures particulates from the fluid before it exits the drip chamber. In some implementations, the predetermined surface area of the filter is between 500 cm2 and 1000 cm2. In some implementations, the filter includes pore sizes between 20-80 microns. In some implementations, the filter includes pore sizes between 180-200 microns.

In some implementations, the IV set further includes a third portion of tubing and a hand pump including an inlet fluidically coupled to the second portion of tubing and an outlet fluidically coupled to the third portion of tubing. In some implementations, the third portion of tubing is a predetermined length and radius. The hand pump includes a first portion, a second portion adjacent to the first portion, and a third portion adjacent to the second portion. The first, second, and third portions have respective heights and respective radii. The second portion has a height and a radius greater than the respective heights and radii of the first and third portions. In some implementations, the hand pump is lantern shaped body.

In some implementations, the hand pump is formed an antistatic material. In some implementations, the first and third portions have the same respective heights and the same respective radii. In some implementations, the hand pump includes another filter configured to capture particulates from the fluid before it exits the hand pump. In some implementations, the hand pump comprises a resilient material such that the hand pump body rebounds to an uncompressed state after it is compressed. In some implementations, the hand pump, when compressed, accelerates a flow of the fluid to at least 13 L/hr.

Note that the various implementations described above can be combined with other implementations described herein (e.g., keep an impedance for a single or group of neuromuscular-signal sensors can be combined with matching of impedances, such that impedances can be both matched and be kept within particular ranges of impedance values). The features and advantages described in the specification are not all inclusive and, in particular, additional features and advantages will be apparent to one of ordinary skill in the art in view of the drawings, specification, and claims. Moreover, it should be noted that the language used in the specification has been principally selected for readability and instructional purposes.

### BRIEF DESCRIPTION OF THE DRAWINGS

So that the present disclosure can be understood in greater detail, a more particular description may be had by reference to the features of various implementations, some of which are illustrated in the appended drawings. The appended drawings, however, merely illustrate pertinent features of the present disclosure. The description may admit to other effective features as the person of skill in this art will appreciate upon reading this disclosure.

FIG. 1 illustrates an IV pump administering a medical fluid to a patient.

FIG. 2 illustrates a conventional IV set 200.

FIGS. 3A-3D illustrate a modified drip chamber, in accordance with some implementations.

FIG. 4 illustrates a modified hand pump of an IV set, in accordance with some implementations.

FIG. 5 illustrates another IV set with an adjusted length, in accordance with some implementations.

FIG. 6 is a conceptual diagram illustrating an example electronic system for controlling a pump, according to aspects of the subject technology.

In accordance with common practice, the various features illustrated in the drawings are not drawn to scale. Accordingly, the dimensions of the various features may be arbitrarily expanded or reduced for clarity. In addition, some of the drawings may not depict all of the components of a given system, method or device. Finally, like reference numerals denote like features throughout the specification and figures.

### DETAILED DESCRIPTION

Numerous details are described herein in order to provide a thorough understanding of the example implementations illustrated in the accompanying drawings. However, some implementations may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the implementations described herein.

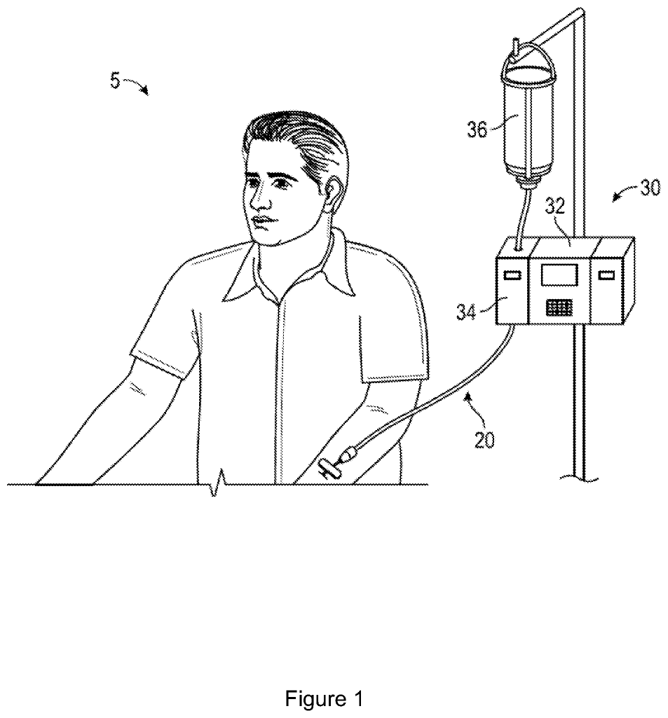

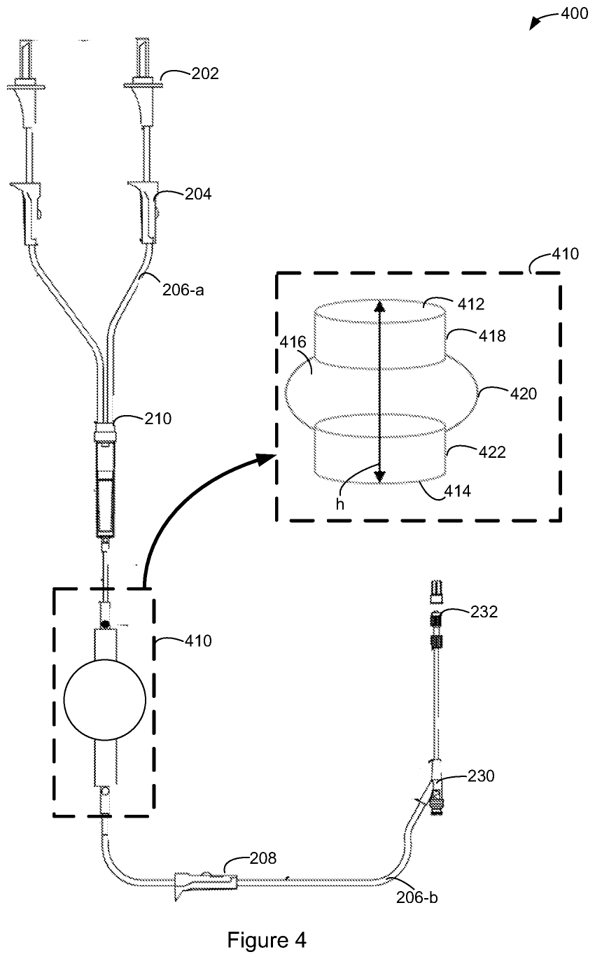

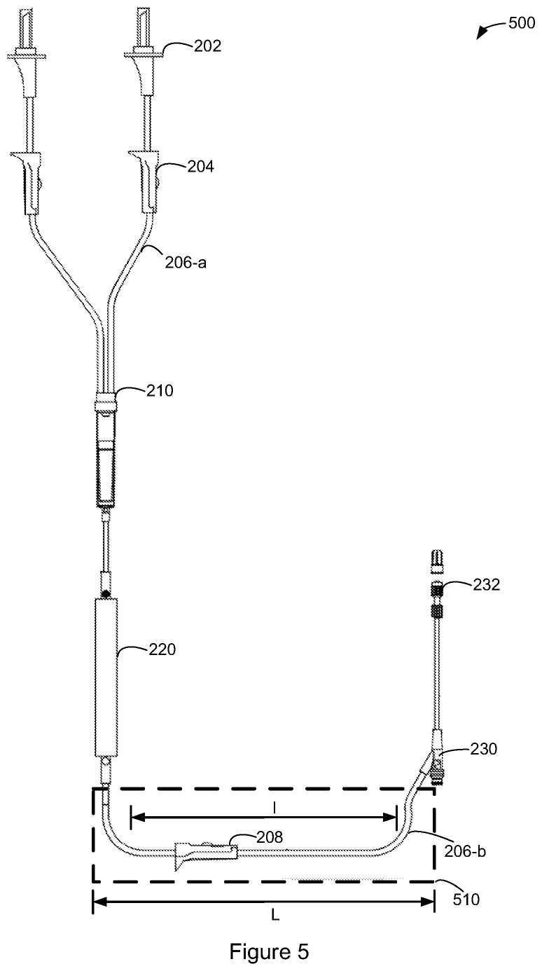

FIG. 1 illustrates an IV pump administering a medical fluid to a patient. The IV pump 30 includes a controller 32 and one or more pump modules 34. An IV set 20 is connected between a container 36 (e.g., an IV bag) of the medical fluid and the patient 5. During operation, IV pump 30 delivers the medical fluid to the patient 5. The IV pump 30 is configured to administer the medical fluid to the patient at a predetermined and uniform rate. In some implementations, the IV set 20 is utilized to further accelerate a flow of medical fluid from the container 36 (or the IV pump 30) to the patient. For example, as shown and described below in reference to FIGS. 4 and 5, the IV set 20 a hand pump can be used to further accelerate the flow of medical fluid.

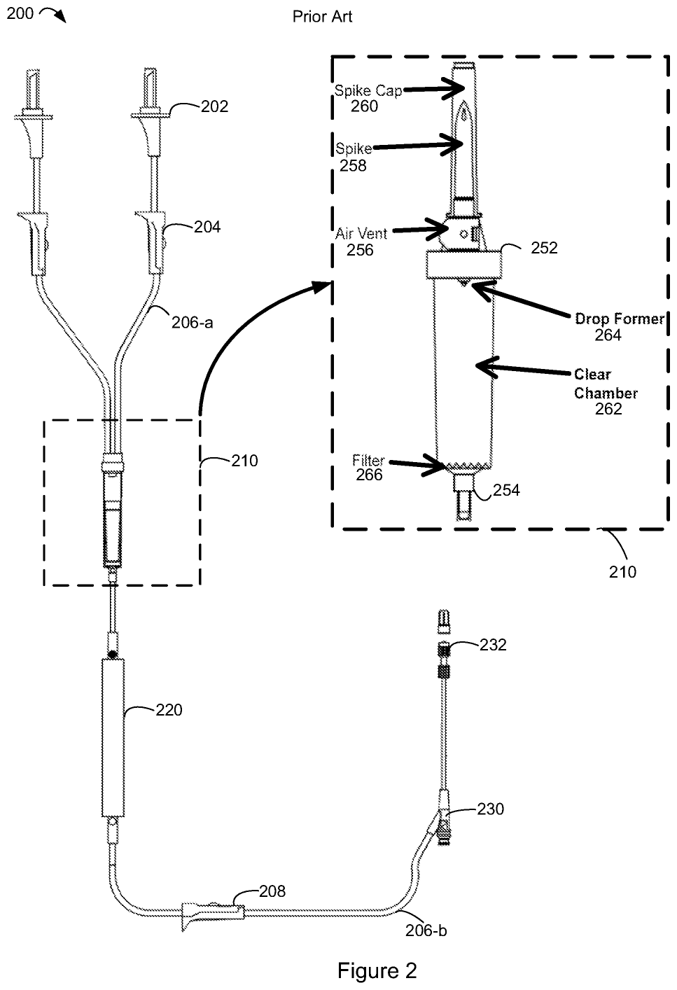

FIG. 2 illustrates a conventional IV set 200. The IV set 200 includes one or more connectors 202, one or more clamps 204 and 208, tubing 206, a drip chamber 210, a hand pump 220, an injection site 230, and an outlet port 232. As described above in reference to FIG. 1, the IV set 200 delivers fluid from a fluid source, such as a container 36, to a patient 5 through tubing 206. More specifically, fluid from the fluid source is introduced into a first portion of the tubing 206-a of the IV set 200 and delivered to the patient 5 (e.g., via a flow generated by an IV pump 30 (FIG. 1) and/or a hand pump 220). The connectors 202 facilitate coupling and/or fluid communication between the first portion of the tubing 206-a and a fluid source (e.g., one or more containers 36). More specifically, the connectors 202 fluidically couple the first portion of the tubing 206-a to one or more one or more containers 36. In some implementations, the connectors 202 can be connector spikes that pierce a membrane of the container 36 to permit fluid communication from the container 36 into the tubing 206. Alternatively, in some implementations, the connectors 202 can be needleless connectors to avoid inadvertent piercing of membranes when fluidically coupling the first portion of the tubing 206-a and the fluid source. In some implementations, the needleless connectors can include a no-drip feature to prevent leaks or surface contamination. In some implementations, the needleless connectors can further include a luer lock to prevent accidental discharges. In some implementations, the needleless connectors engage with fluid containers that include mating connectors.

In some implementations, a second portion of the tubing 206-b is coupled to the patient 5 via the outlet port 232. Additional medical fluids or treatments can be introduced to the patient via the IV set 200. In some implementations, additional medical fluids or treatments can be introduced into the IV set 200 via the injection site 230. In some implementations, the fluid container allows for pulling aliquots of blood for analysis.

Clamps 204 and 208 are configured to control fluid flow through the tubing 206 of the IV set 200. For example, clamps 204 and 208 can occlude the flow of fluid through the tubing 206 such that no fluid is distributed to the patient 5. Alternatively, clamps 204 and 208 can partially occlude the flow of fluid through the tubing 206 such that the flow of fluid distributed to the patient 5 is reduced. The clamps 204 and 208 can be roller clamps, pinch clamps, slide clamps, and/or other clamps known in the art.

In some implementations, the drip chamber 210 is formed of a transparent material or a semi-transparent material to provide a visual indicator of the flow rate of a medical fluid therethrough (e.g., clear chamber 262). The drip chamber 210 is configured to allow clinicians and/or other medical practitioners to monitor and adjust a flow rate of the medical fluid based on the visual indicator (e.g., clear chamber 262) provided by the drip chamber 210 (e.g., counting the number of drops per second). During operation, medical fluid can drip or otherwise flow through the chamber volume of the drip chamber 210. Medical fluid can enter the drip chamber 210 through an upper portion or inlet portion 252 defined in the drip chamber 210. The inlet portion 252 is in fluid communication with the first portion of tubing 206-a. Fluid flow exits the drip chamber 210 through a lower portion or outlet portion 254. The outlet portion 254 can be in fluid communication with the second portion of the tubing 206-b.

Conventional drip chambers include a spike 258 (or needle) to fluidically couple a drip chamber 210 to tubing 206 and/or a fluid source (e.g., container 36). In some implementations, the spike 258 pierces a membrane of the container 36 to permit fluid communication from the container 36 into the tubing 206. In some implementations, the spike 258 is covered by spike cap 260. The spike cap 260 is configured to prevent damage from the spike 258, keep the spike 258 clean, and/or prevent injury caused by the spike 258 accidentally making contact with a person's body. In some implementations, the spike 258 is coupled to an air vent 256. The air vent 256 is configured to expel air from the drip chamber 210. The spike 258 and air vent 256 are fluidically coupled to the inlet portion 252 of the drip chamber 210. In some implementations, the inlet portion 252 of the drip chamber 210 is fluidically coupled to a drop former 264. The drop former 264 is configured to generate (or form) one or more droplets that are used by clinicians and/or other medical practitioners to monitor a flow rate of the medical fluid (e.g., by counting the number of drops per second).

In some implementations, the drip chamber 210 includes a filter 266 for filtering the medical fluid passing therethrough. In some implementations, the filter 266 is disposed within the drip chamber 210. In some implementations, the filter 266 is disposed within the inlet portion 252 or the outlet portion 254 of the drip chamber 210. Alternatively, in some implementations, the filter 266 is disposed within the chamber volume of the drip chamber 210. In some implementations, the filter 266 is an integral part of the drip chamber 210. During operation, fluid can flow through the inlet portion 252 of the drip chamber 210, through a filter 266 to an outlet portion 254 of the drip chamber 210. As medical fluid flows through the drip chamber 210, the fluid can be filtered prior to flowing out of the drip chamber 210 and through the second portion of tubing 206-b. Fluid within the chamber volume of the drip chamber 210 can pass through the filter 266 to prevent the transfer of bacteria, microorganisms, and/or other pathogens to the patient. As can be appreciated, a positive pressure differential can direct fluid flow through the filter 266. As described herein, the filter 266 can selectively filter the flow through the drip chamber 210. The filter 266 can have an average filter opening ranging between 15 to 200 microns. In some implementations, the average filter opening can range between 180 to 200 microns. Optionally, the filter 266 can have pores that vary in size.

During operation, the filter 266 can become clogged with particulate, limiting the filtering efficiency and flow through the filter 266. The conventional IV set 200 can include a hand pump 220 to dislodge sediment or particulate embedded in the filter 266, extending the life of the filter 266 and increasing the flow through the filter 266. The hand pump 220 can direct back flow or back pressure through the filter 266 to dislodge particulate embedded in the filter 266. In the depicted example, a clinician can actuate the hand pump 220 to create back flow through the filter 266.

In some implementations, the drip chamber 210 can equalize pressure differentials between the chamber volume and the environment during operation. In some implementations, the drip chamber 210 can be formed from a resilient material to allow the drip chamber 210 to be squeezed or compressed to draw in medical fluid for priming of an IV system. In some implementations, the drip chamber 210 draws in medical fluid for priming the IV system. As can be appreciated, the drip chamber 210 can be filled with a desired volume of medical fluid during the priming operation.

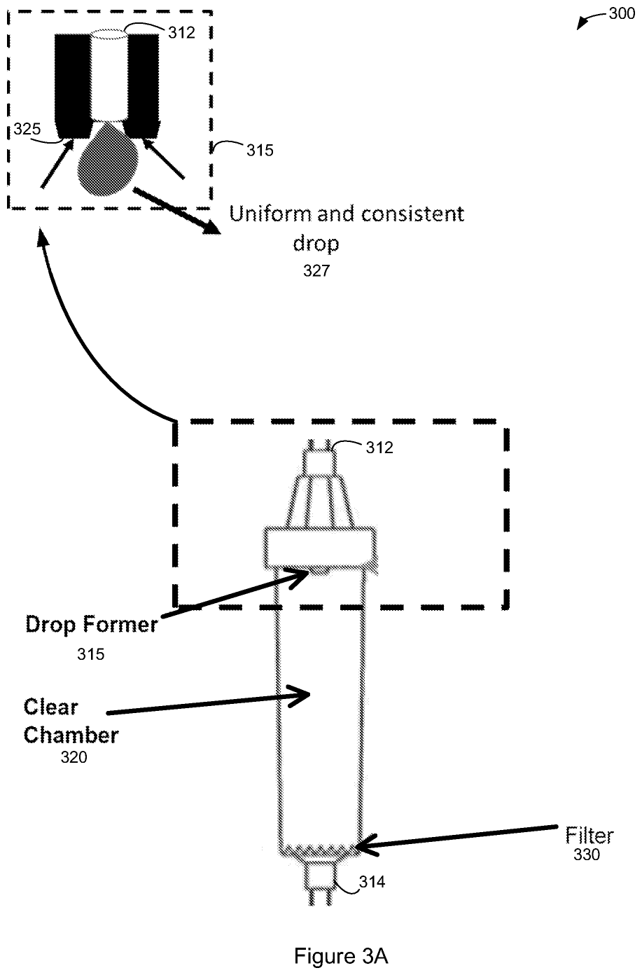

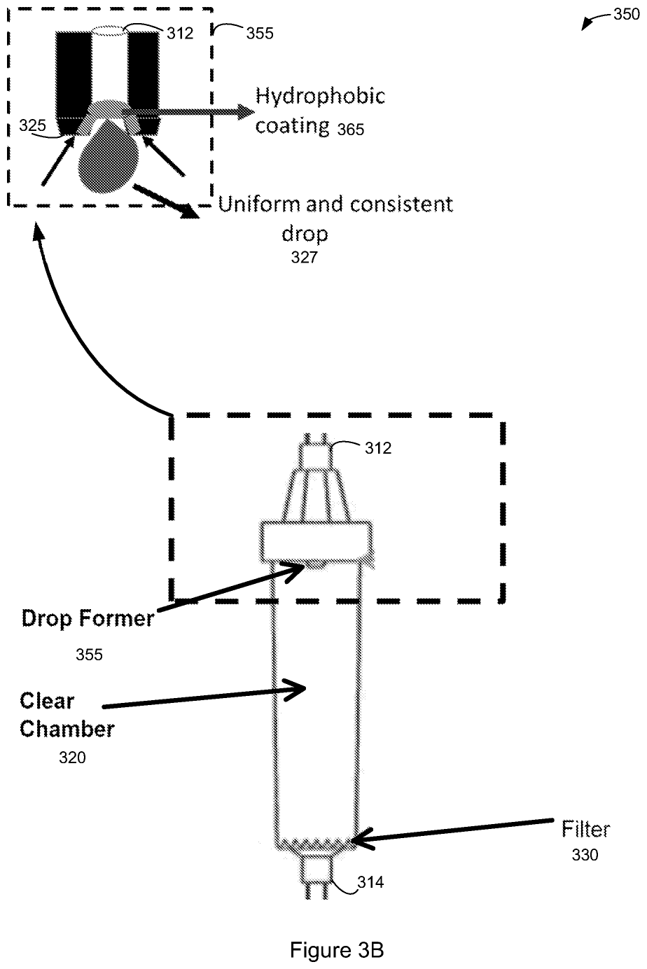

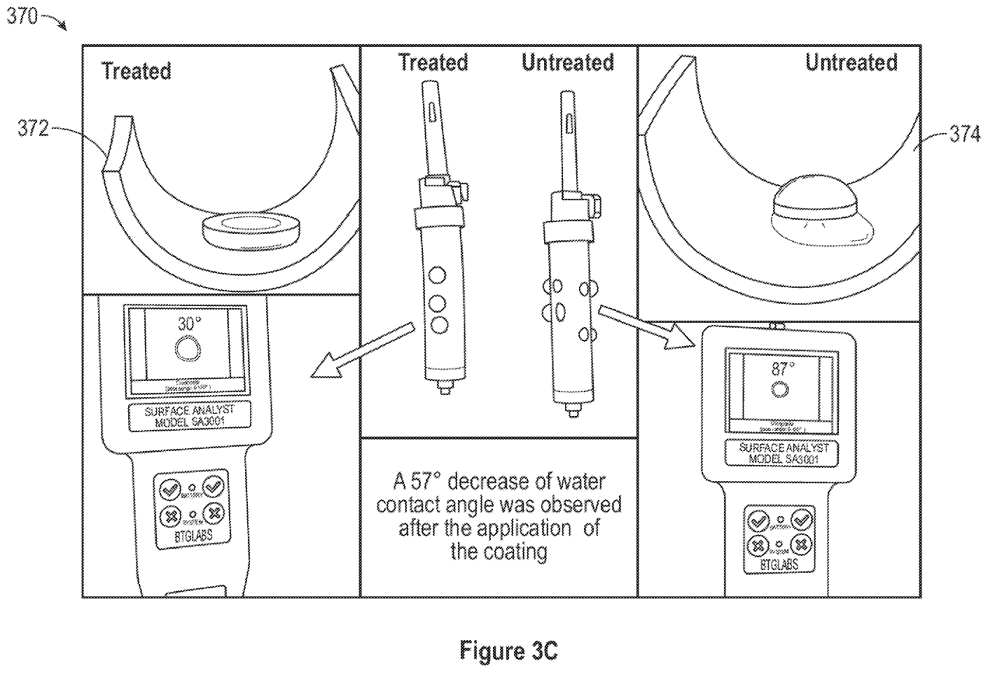

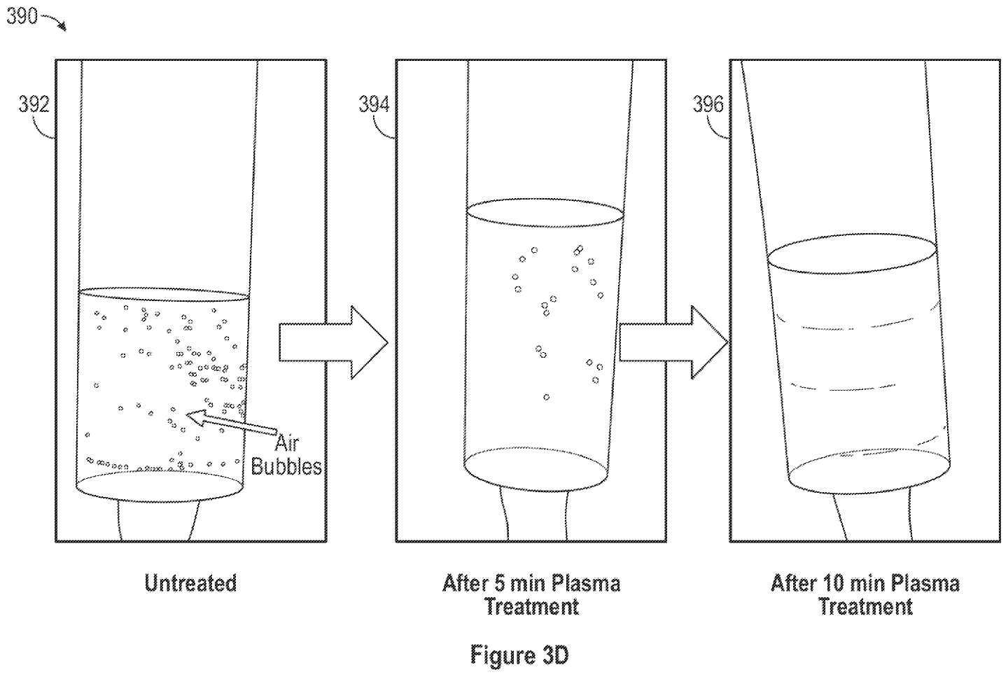

FIGS. 3A-3D illustrate a modified drip chamber, in accordance with some implementations. Modified drip chamber 300 is configured to allow clinicians and/or other medical practitioners to monitor and adjust a flow rate of the medical fluid based on the visual indicator as described above in reference to drip chamber 210 in FIG. 2. The modified drip chamber 300 includes at least a spikeless inlet portion 312, a modified drop former 315, a coated clear chamber 320, a modified filter 330, and an outlet portion 314. The modified drip chamber 300 is configured to increase the flow rate and accuracy of fluid through the modified drip chamber 300, increase the accuracy and consistency of the droplets generated by the drop former 315, and increase the overall volume that can be held within the clear chamber 320 relative to a conventional drip chamber 210 (FIG. 2).

The spikeless inlet portion 312 (e.g., similar to an inlet 252 without a spike 258 fluidically connected thereto as shown and described in reference to FIG. 2) is configured to allow the modified drip chamber 300 to be fluidically coupled lower on an IV set 200. Lower on the IV set 200 means, for purposes of this disclosure, between chest and waist level of a clinician (or medical practitioner) such that the clinician does not have to reach to adjust or interact with the modified drip chamber 300. By allowing the modified drip chamber 300 to be fluidically coupled lower on the IV set 200, the spikeless inlet portion 312 improves the ease of use of the modified drip chamber 300 and reduces clinician fatigue (relative to the conventional drip chamber 210).

The modified drop former 315 includes a curved surface 325. The modified drop former 315 is fluidically coupled to the spikeless inlet portion 312. The curved surface 325 of the modified drop former 315 is configured to improve the drop accuracy. More specifically, the curved surface 325 of the modified drop former 315 generates uniform and consistent droplets (e.g., droplet 327) that can be monitored by clinicians. In addition, in some implementations, the curved surface 325 of the modified drop former 315 is configured to increase the size of the generated droplets such that a flow rate of the modified drop former 315 is improved (e.g., the flow rate is increased relative to the conventional drop former 264).

The modified filter 330 is configured to filter medical fluid passing therethrough as described above in reference to FIG. 2. The modified filter 330 includes an increased surface area relative to filter 266. In particular, the modified filter 330, in some implementations, has a surface area of 500 cm² to 1000 cm², which is approximately 10 times greater than a surface area of the filter 266 (e.g., 20 cm² to 50 cm²). In some implementations, the modified filter 330 is formed of a Polyethersulfone (PES) membrane. In some implementations, the modified filter 330 is one of a blood and blood clot filtration unit, non-vented blood set filtration unit. The modified filter 330 can have an average filter opening ranging between 20 to 80 microns. In some implementations, the average filter opening of the modified filter 330 is between 180 to 200 microns. Optionally, the modified filter 330 can have pores that vary in size. The modified filter 330 is a high throughput filtration unit such that there is an increased flow rate and improved efficiency. In some implementations, the modified filter 330 is fluidically coupled to the outlet portion 314. In some implementations, an activation force of the modified filter 330 (or the modified drip chamber 300) is increased (relative to a conventional filter 266 and/or drip chamber 210; FIG. 2) such that a flow rate in the tubing 206 is further increased.

In some implementations, the coated clear chamber 320 is formed from a resilient or deformable material to allow the coated clear chamber 320 to be squeezed or compressed to draw in fluid for priming of an IV system. In some implementations, the resilient or deformable material includes silicone, rubber, flexible PVC grade material, thermoplastic elastomers (e.g., Styrene thermoplastic elastomer), plastics, polyurethane, and/or other flexible material that can be squeezed and return to its resting state. The coated clear chamber 320 is treated (e.g., application of a coat) to decrease the surface tension and/or increase the wettability on the side walls of the coated clear chamber 320 by eliminating air bubbles (as shown and described below in reference to FIGS. 3C and 3D. In some implementations, the coated clear chamber 320 decreases a fluid contact angle by least 55 degrees (e.g., as shown in FIG. 3C, the contact angle was decreased by at least 57 degrees). In some implementations, the coated clear chamber 320 is coated such that a coefficient of friction is increased so that a medical fluid (e.g., blood cells) slide through and do not stick to the walls of the coated clear chamber 320. In some implementations, the coated clear chamber 320 is treated with an antistatic and blood compatible material. By decreasing the surface tension and increasing the wettability on the side walls of the clear chamber 320, a greater amount of fluid can be held in the clear chamber 320 and the flow rate through the clear chamber 320 can be increased.

FIG. 3B illustrates an additional modified drip chamber, in accordance with some implementations. The additional modified drip chamber 350 is an instance of the modified drip chamber 300 that further includes a hydrophobic coating 365 on the curved surface 325. The hydrophobic coating 365 is placed on the sides of the drop former 355 exit to increase the contact angle of the medical fluid leaving the drop former 355 to form uniform and consistent droplets.

FIG. 3C illustrates a water contact angle of a fluid (e.g., water) on a treated surface 372 of a modified drip chamber 300 (e.g., coated clear chamber 320) compared to an untreated surface 374 of a drip chamber 210 (e.g., clear chamber; FIG. 2). As shown in FIG. 3C, the water contact angle of a treated surface 372 is decreased to an angle of at least 57 degrees compared to an untreated surface 374 of a clear chamber. The decreased water contact angle lowers the number of failures; improves accuracy of the measured volume of the coated clear chamber 320; and/or reduces the occurrence of occlusions by decreasing the surface tension of a surface of the coated clear chamber 320 and increasing wettability on the side walls of the modified drip chamber 300 (e.g., by eliminating air bubbles within the coated clear chamber 320).

FIG. 3D illustrates different clear chamber coatings, in accordance with some implementations. In particular, an untreated clear chamber 392, a 5 minute coated clear chamber 394, and a 10 minute coated clear chamber 396. As shown by the untreated clear chamber 392, air bubbles build up on the surface of the untreated clear chamber 392. The air bubbles on the surface of the untreated clear chamber 392 can result in failures within a drip chamber, inaccurate volumes readings, and/or increase the number of failures in a drip chamber. Alternatively, a clear chamber treated for at least 5 minutes (as shown by the 5 minute coated clear chamber 394) reduces the number of air bubbles formed within a drip chamber. Further, a clear chamber treated for at least 10 minutes (as shown by the 10 minute coated clear chamber 396) removes substantially all air bubbles within a drip chamber.

FIG. 4 illustrates a modified hand pump of an IV set, in accordance with some implementations. IV set 400 includes one or more features described above in reference to FIG. 2. For example, the IV set 400 includes one or more connectors 202, one or more clamps 204 and 208, tubing 206, a drip chamber 210, an injection site 230, and an outlet port 232. The IV set 400 distributes medical fluid to a patient as described above in reference to FIG. 2. The IV set 400 further includes a modified hand pump 410 to accelerate delivery of a fluid (i.e., increase flow of the fluid) to a patient 5 (FIG. 1) and reduce clinician (or other medical practitioner) fatigue, which allows for extended use of the IV set 400. In some implementations, the modified hand pump 410 is configured to increasing a volume and flow of a fluid through the IV set 400 to approximately 13-15 liters/hour (where approximately means+/−0.5 liters/hour).

In some implementations, the modified hand pump 410 is formed of a transparent material or a semi-transparent material to provide a visual indicator of the flow rate of fluid therethrough. Clinicians (or medical practitioners) can monitor and adjust the flow rate of the fluid based on the visual indicator provided by the modified hand pump 410 (e.g., a transparent material or a semi-transparent material that allows the clinicians to visually inspect the fluid flowing through the modified hand pump 410). During operation, fluid can drip or otherwise flow through the modified hand pump 410. Fluid can enter the modified hand pump 410 through upper portion or inlet portion 412 defined by the modified hand pumps 410. The inlet portion 412 is in fluid communication with the first portion of the tubing 206-a. Fluid flow can exit the modified hand pump volume 416 of the modified hand pump 410 through lower portion or outlet portion 414. The outlet portion 414 can be in fluid communication with a second portion of the tubing 206-b.

The modified hand pump 410 has a substantially cylindrical body. In some implementations, the modified hand pump 410 is formed of at least three continuous portions of tubing. In some implementations, the at least three continuous portions of tubing include a second portion of tubing 420 between a first portion of tubing 418 and a third portion of tubing 422. In some implementations, the first portion of tubing 418 forms, in part, the inlet portion 412 of the modified hand pump 410 and the second portion of tubing 422 forms, in part, the outlet portion 414 of the modified hand pump 410. In some implementations, the first portion of tubing 418 and the third portion of tubing 422 are the same height and have the same radius. Alternatively, in some implementations, the first portion of tubing 418 and the third portion of tubing 422 have distinct heights and/or radii. The second portion of tubing 420 includes a radius that is greater than the respective radii of the first portion of tubing 418 and the third portion of tubing 422.

In some implementations, the second portion of tubing 420 includes a radius that varies across its height (e.g., height “h” of the modified hand pump 410). In some implementations, the second portion of tubing 420 is substantially symmetrical with the radius of the second portion of tubing 420 being the greatest at the center (e.g., half of its height). In some implementations, the height of the modified hand pump 410 is less than the height of hand pump 220. In some implementations, the modified hand pump 410 forms a lantern shaped body. The lantern shaped body increases the total volume of the modified hand pump 410 and provides a larger diameter of inlet and outlet diameter relative to conventional hand pumps (e.g., hand pump 220; FIG. 2). Further, the lantern shaped body provides an ergonomic body that can be conveniently actuated (e.g., squeezed or compressed) to accelerate fluid from the modified hand pump 410 while reducing clinician fatigue.

In some implementations, the modified hand pump 410 is formed from a resilient or deformable material to allow the modified hand pump 410 to be squeezed or compressed to draw in fluid for priming of an IV system. In addition, in some implementations, the modified hand pump 410 is actuated to accelerate fluid from the modified hand pump 410 volume 416 to the second portion of the tubing 206-b. In some implementations, the resilient or deformable material includes silicone, rubber, flexible PVC grade material, thermoplastic elastomers (e.g., Styrene thermoplastic elastomer), plastics, polyurethane, and/or other flexible material that can be squeezed and return to its resting state.

In some implementations, the modified hand pump 410 equalizes pressure differentials between the hand pump volume 416 and the environment during operation. In some implementations, by reducing the hand pump volume 416 (e.g., by squeezing the modified hand pump 410), the fluid within the hand pump volume 416 is pressurized. The pressurized fluid within the hand pump volume 416 can be accelerated to distribute the fluid to the user at a faster rate. Advantageously, due to the hand pump volume 416, the modified hand pump 410 improve flow through the IV set 400 rapidly while reducing the strain and fatigue on clinicians (or medical practitioners). In some implementations, the modified hand pump 410 draws in fluid for priming the IV system. For example, the hand pump volume 416 can be filled with a desired volume of fluid during the priming operation.

In some implementations, the modified hand pump 410 includes a filter analogous to filter 266 or modified filter 330 described above in reference to FIGS. 2-3D. More specifically, similar to the drip chamber 210 and modified drip chamber 300, a filter of the modified hand pump 410 allows for filtration of fluid passing therethrough. In some implementations, the modified hand pump 410 integrates a filter. In some implementations, due to the increased size and volume of the modified hand pump 410, the filter disposed within the modified hand pump 410 is larger and has more surface area than filter 266 or modified filter 330 utilized within the drip chamber 210 or the modified filter 330, respectively. In some implementations, the filter of the modified hand pump 410 can be any suitable size. In some implementations, the modified hand pump 410 includes a filter that is used in place of the filter 266 or the modified filter 330. Alternatively, in some implementations, the modified hand pump 410 with a filter is used in place of the drip chamber 210 filter 266 or the modified drip chamber 300.

In some implementations, during operation, as fluid flows through the modified hand pump 410, the fluid is filtered prior to flowing out of the modified hand pump 410 and into the second portion of the tubing 206-b. As described above, the filter to prevents the transfer of bacteria, microorganisms, and/or other pathogens to the patient. During operation (i.e., the administration of medical fluid to the patient), the filter(s) can be clogged with particulate, reducing flow through an IV system. In some implementations, the modified hand pump 410 is used to agitate any particulate trapped within a filter (e.g., filter 266, modified filter 330, or an integrated filter) or other portions of the IV set 400, increasing flow through the filter and the IV set 400. In other words, the modified hand pump 410 accelerates the flow of medical fluid and/or agitate any particulate trapped within one or more filters via the application of a force to the modified hand pump 410 (e.g., squeezing the plurality of hand pumps).

In addition to accelerating a flow of fluid, the pressurized fluid within the hand pump volume 416 is such that, upon actuation or compression of the modified hand pump 410, back flow is forced through filters of the modified hand pump 410 (or in other portions of the IV set 400), displacing or dislodging particulate from a filter. Advantageously, due to the hand pump volume 416, the modified hand pump 410 improves flow through the IV set 400 rapidly while reducing the strain and fatigue on clinicians (or medical practitioners) by providing an easy-to-use ergonomic body.

FIG. 5 illustrates another IV set with an adjusted length, in accordance with some implementations. IV set 500 includes one or more features described above in reference to FIGS. 3A-4. For example, the IV set 500 includes one or more connectors 202, one or more clamps 204 and 208, a hand pump 220, tubing 206, a drip chamber 210, an injection site 230, and an outlet port 232. The IV set 500 is configured to distributes medical fluid to a patient as described above in reference to FIGS. 3A-4. The IV set 500 further includes a shortened tubing 206. More specifically, in some implementation, s the length of a second portion of the tubing 206-b is decreased. In some implementations, a portion of tubing 206 from the hand pump 220 to the injection site 230 is shortened (e.g., represented by length “L”). Alternatively, in some implementations, a portion of tubing 206 fluidically coupled to clamp 208 is shortened. Additionally, in some implementations, a radius of the portion of tubing 206 from the hand pump 220 to the injection site 230 (or the portion of tubing 206 fluidically coupled to clamp 208) is increased. In some implementations, the shortened length and/or the increased radius of the second portion of the tubing 206-b increase the flow rate of the fluid within the IV set 500. In some implementations, a volume and flow rate of the fluid is increased to greater that 15 liters per hour.

IV sets consistent with the present disclosure may include all or at least some shown and described above in reference to FIGS. 3A-5. In some implementations, one or more components (e.g., modified drip chamber 300, modified hand pump 410, etc.) of an IV set can be coated (e.g., with a hydrophobic coating) to increase a coefficient of friction such that fluid does not stick to one or more walls of the IV set and a flow rate within the one or more components is increased. In some implementations, the coating is an antistatic and blood compatible material.

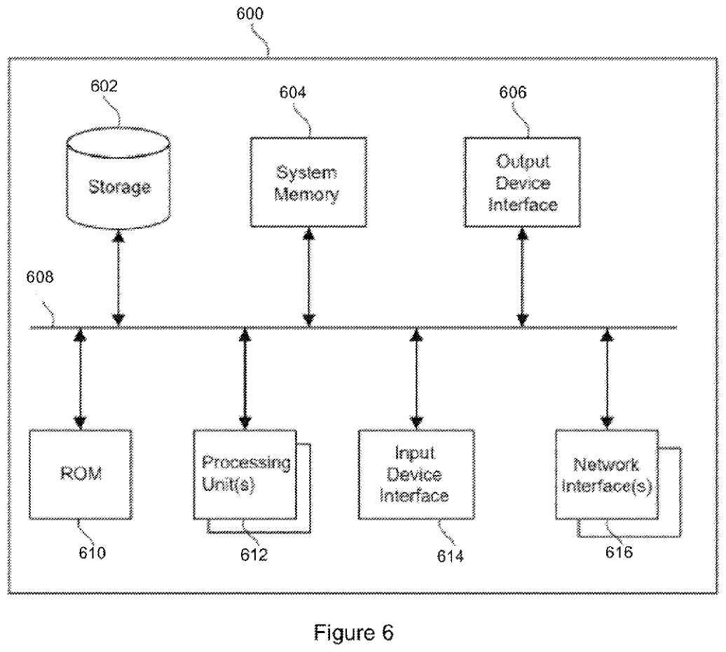

FIG. 6 is a conceptual diagram illustrating an example electronic system 600 for controlling a pump, according to aspects of the subject technology. Electronic system 600 may be a specifically configured computing device for execution of software associated with components and processes provided by FIGS. 1 through 5, including but not limited to controller 32 of IV pump 30. Electronic system 600 may be representative, in combination with the disclosure regarding FIGS. 1 through 5.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as a random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations. Such storage and/or memory devices 602, 604 may be representative of memory of controller 32.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”), such as those shown in controller 32 of FIG. 1. Output device interfaces 606 (e.g., shown as a display in the controller 60 of FIG. 1) enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An IV set including a first portion of tubing, a second portion of tubing, and a drip chamber. The drip chamber includes a spikeless inlet fluidically coupled to the first portion of tubing, an outlet fluidically coupled to the second portion of tubing, and a curved drop former positioned within the drip chamber between the inlet and the outlet, and configured to receive a fluid via the spikeless inlet and form, based on a curvature of its surface, one or more droplets having a substantially uniform consistency and flowing at a substantially constant rate through the drip chamber.

Clause 2. The IV set of Clause 1, the drip chamber further comprises a filter of a predetermined surface area fluidically coupled to the outlet such that the filter captures particulates from the fluid before it exits the drip chamber.

Clause 3. The IV set of Clause 2, the predetermined surface area of the filter is between 500 cm² and 1000 cm².

Clause 4. The IV set of any of Clause 2 and Clause 3, the filter includes pore sizes between 20-80 microns.

Clause 5. The IV set of any of Clause 2 and Clause 3, the filter includes pore sizes between 180-200 microns.

Clause 6. The IV set of any of Clause 1 through Clause 5, curved drop former includes a hydrophobic coating.

Clause 7. The IV set of any of Clause 1 through Clause 6, a body of the drip chamber includes a hydrophobic coating that forms a predetermined contact angle such that the one or more droplets are uniform and consistent.

Clause 8. The IV set of any of Clause 1 through Clause 7, the IV set further including a third portion of tubing, and a hand pump including an inlet fluidically coupled to the second portion of tubing and an outlet fluidically coupled to the third portion of tubing, the hand pump including a first portion, a second portion adjacent to the first portion, and a third portion adjacent to the second portion. The first, second, and third portions have respective heights and respective radii, the second portion having a height and a radius greater than the respective heights and radii of the first and third portions.

Clause 9. The IV set of Clause 8, the hand pump is formed an antistatic material.

Clause 10. The IV set of any of Clause 8 and Clause 9, the first and third portions have the same respective heights and the same respective radii.

Clause 11. The IV set of any of Clause 1 through Clause 10, the hand pump includes another filter configured to capture particulates from the fluid before it exits the hand pump.

Clause 12. The IV set of any of Clause 1 through Clause 11, the third portion of tubing is a predetermined length and radius.

Clause 13. The IV set of any of Clause 8 through Clause 12, the hand pump, when compressed, accelerates a flow of the fluid to at least 13 L/hr.

Clause 14. The IV set of any of Clause 8 through Clause 13, the hand pump comprises a resilient material such that a body of the hand pump rebounds to an uncompressed state after it is compressed.

Clause 15. The IV set of any of Clause 1 through Clause 14, the drip chamber is formed an antistatic material.

Clause 16. The IV set of any of Clause 1 through Clause 15, the drip chamber comprises a resilient material such that the drip chamber rebounds to an uncompressed state after it is compressed.

### Further Consideration

In some implementations, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

It will be understood that, although the terms “first,” “second,” etc. may be used herein to describe various elements, these elements should not be limited by these terms. These terms are only used to distinguish one element from another. Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

The terminology used herein is for the purpose of describing particular implementations only and is not intended to be limiting of the claims. As used in the description of the implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such an implementation may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An IV set, comprising:
a first portion of tubing;
a second portion of tubing; and
a drip chamber including:
a spikeless inlet fluidically coupled to the first portion of tubing,
an outlet fluidically coupled to the second portion of tubing, and
a curved drop former positioned within the drip chamber between the inlet and the outlet, and configured to receive a fluid via the spikeless inlet and form, based on a curvature of its surface, one or more droplets having a substantially uniform consistency and flowing at a substantially constant rate through the drip chamber.

2. The IV set of claim 1, wherein the drip chamber further comprises a filter of a predetermined surface area fluidically coupled to the outlet such that the filter captures particulates from the fluid before it exits the drip chamber.

3. The IV set of claim 2, wherein the predetermined surface area of the filter is between 500 cm² and 1000 cm².

4. The IV set of claim 2, wherein the filter includes pore sizes between 20-80 microns.

5. The IV set of claim 2, wherein the filter includes pore sizes between 180-200 microns.

6. The IV set of claim 1, wherein the curved drop former includes a hydrophobic coating.

7. The IV set of claim 1, wherein a body of the drip chamber includes a hydrophobic coating that forms a predetermined contact angle such that the one or more droplets are uniform and consistent.

8. The IV set of claim 1, further comprising:
a third portion of tubing; and
a hand pump including an inlet fluidically coupled to the second portion of tubing and an outlet fluidically coupled to the third portion of tubing, the hand pump including a first portion, a second portion adjacent to the first portion, and a third portion adjacent to the second portion, wherein the first, second, and third portions have respective heights and respective radii, the second portion having a height and a radius greater than the respective heights and radii of the first and third portions.

9. The IV set of claim 8, wherein the hand pump is formed an antistatic material.

10. The IV set of claim 8, wherein the first and third portions have the same respective heights and the same respective radii.

11. The IV set of claim 8, wherein the hand pump includes another filter configured to capture particulates from the fluid before it exits the hand pump.

12. The IV set of claim 8, wherein the third portion of tubing is a predetermined length and radius.

13. The IV set of claim 8, wherein the hand pump, when compressed, accelerates a flow of the fluid to at least 13 L/hr.

14. The IV set of claim 8, wherein the hand pump comprises a resilient material such that a body of the hand pump rebounds to an uncompressed state after it is compressed.

15. The IV set of claim 1, wherein the drip chamber is formed an antistatic material.

16. The IV set of claim 1, wherein the drip chamber comprises a resilient material such that the drip chamber rebounds to an uncompressed state after it is compressed.
